(19) United States
(12) Patent Application Publication
(10) Pub. No.: US 2005/0288283 A1
(43) Pub. Date: Dec. 29, 2005

(54) Topical formulations for treating allergic diseases

(75) Inventor: Mark R. Hellberg, Arlington, TX (US)

Correspondence Address: Alcon Research, Ltd.;Patrick M. Ryan(Q-148), IP Legal Department, 6201 So. Freeway, Fort Worth, TX, 76134-2099, US

(73) Assignee: Alcon, Inc.

(21) Appl. No.: 11/167,634

(22) Filed: Jun. 27, 2005

Related U.S. Application Data
(60) Provisional application No. 60/583,380, filed on Jun. 28, 2004.

(51) Int. Cl. A61K 31/5377; A61K 31/473

(52) U.S. Cl. 514/232.5; 514/290

(57) ABSTRACT

Methods for topically treating allergic diseases of the eye, ear or nose using tricyclic compounds are disclosed.

This application claims priority to U.S. Provisional Application, Ser. No. 60/583,380 filed Jun. 28, 2004.

### BACKGROUND OF THE INVENTION

1. Field of the Invention

The present invention relates to topical formulations used for treating allergic diseases. More particularly, the present invention relates to topical use of certain tricyclic compounds for treating allergic diseases of the eyes, ears or nose.

2. Description of the Related Art

As taught in U.S. Pat. Nos. 4,871,865 and 4,923,892, both assigned to Burroughs Wellcome Co. (“the Burroughs Wellcome Patents”), certain carboxylic acid derivatives of doxepin, including 11-(3-dimethylaminopropylidene)-6,11-dihydrodibenz[b,e]oxepine-2-carboxylic acid and 11-(3-dimethylaminopropylidene)-6,11-dihydrodibenz[b,e]oxepine-2(E)-acrylic acid, have antihistamine and antiasthmatic activity. These two patents classify the carboxylic acid derivatives of doxepin as mast cell stabilizers with antihistaminic action because they are believed to inhibit the release of autacoids (i.e., histamine, serotonin, and the like) from mast cells and to inhibit directly histamine's effects on target tissues. The Burroughs Wellcome Patents teach various pharmaceutical formulations containing the carboxylic acid derivatives of doxepin; Example 8 (I) in both of the patents discloses an ophthalmic solution formulation.

U.S. Pat. No. 5,116,863 discloses that certain dibenz[b,e,]oxepin derivatives, including the compound 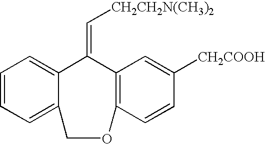
(i.e., Z-11-(3-dimethylaminopropylidene)-6,11-dihydrodibenz[b,e]oxepin-2-acetic acid), have anti-allergic and anti-inflammatory activity. Medicament forms taught by the Kyowa patent for the acetic acid derivatives of doxepin include a wide range of acceptable carriers; however, only oral and injection administration forms are mentioned. In the treatment of allergic eye disease, such as allergic conjunctivitis, such administration methods require large doses of medicine.

U.S. Pat. No. 5,461,805 discloses that 11-(3-dimethylaminopropylidene)-6,11-dihydrodibenz[b,e]oxepin-2-acetic acid and salts are useful in the topical treatment of allergic eye diseases.

What is needed are additional compounds suitable for topical ophthalmic, otic or nasal use that have both anti-histaminic and mast cell stabilizing activity.

### SUMMARY OF THE INVENTION

The present invention provides a method for treating an allergic diseases characterized by topically administering to the eye, ear or nose a formulation which contains a therapeutically effective amount of a tricyclic compound of formula I, II, or III, or a pharmaceutically acceptable salt thereof. The compounds of formulas I, II, and III have both antihistaminic and mast cell stabilizing activity. In a preferred embodiment, the compounds of the present invention are used to treat an allergic eye disease selected from the group consisting of allergic conjunctivitis; vernal conjunctivitis; vernal keratoconjunctivitis; and giant papillary conjunctivitis.

### DETAILED DESCRIPTION OF THE INVENTION

The tricyclic compounds useful in the methods of the present invention are defined by formulas I, II, and III: 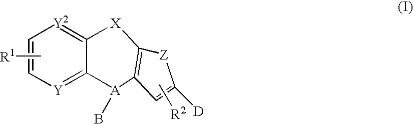
wherein:
Y, Y²═CH;
X═—CH₂—O—;
Z=-CH═CH—;
R¹, R²═Cl, Br, F, CF₃, C₁₋₆ alkyl, C₁₋₆ alkylO—;
A=C, CH, N;
provided that when A=CH or N, then B═CH₂—CH₂—CH₂—Y³, and when A=C, then B═CH—CH₂CH₂—Y³ or 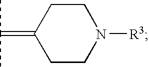
R³═C₁₋₄ alkyl;
Y³═NR⁴R⁵, or 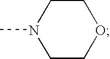
R⁴, R⁵═H, C₁₋₄ alkyl;
D=X¹—(CH₂)ₙ—X²;
X¹=a direct bond;
n=1-6;
X²═NHC(O)R³, NHS(O)₂R³; OC(O)NR⁶R⁷, NHC(O)NR⁶R⁷; and
R⁶, R⁷═H, C₁₋₄ alkyl; 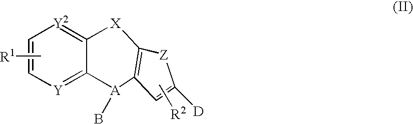
   wherein:
Y, Y²═CH, N;
X═—CH₂—CH₂—, —CH═CH—, CH₂—S—, 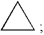
Z=-CH—CH—, —S—;
R¹, R²═Cl, Br, F, CF₃, C₁₋₆ alkyl, C₁₋₆ alkylO—;
A=C, CH, N;
provided that when A=CH or N, then B═CH₂—CH₂—CH₂—Y³;
and when A=C, then B═CH—CH₂CH₂—Y³ or 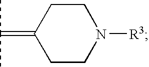
R³═C₁₋₄ alkyl;
Y³═NR⁴R⁵, or 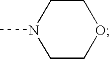
R⁴, R⁵═H, C₁₋₄ alkyl;
Y═X¹—(CH₂)ₘ—X²;
X¹═O, a direct bond;
n=1-6, provided that when X¹ is O, then n=2-6;
X²═H, OH, OR³, OC(O)R³, NHC(O)R³, NHS(O)₂R³, OC(O)NR⁶R⁷, NHC(O)NR⁶R⁷, C(O)NR⁸R^{9,}
R⁶, R⁷═H, C₁₋₄ alkyl;
R⁸, R⁹═H, C₁₋₄ alkyl, OH, OCH₃, provided that only one of R⁸ and R⁹ can be OH or OCH₃; 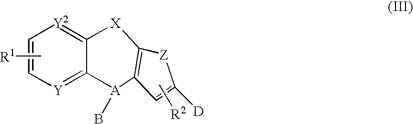
   wherein:
Y, Y²═CH, N, provided that at least one of Y and Y² is N;
X═CH₂—O;
Z=-CH—CH—, —S—;
R¹, R²═Cl, Br, F, CF₃, C₁₋₆alkyl, C₁₋₆alkylO—;
A=C, CH, N;
provided that when A=N, then B═CH₂—CH₂—CH₂—Y³, and when A=CH then B═ 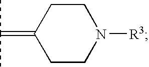
R³═C₁₋₄ alkyl;
Y³═NR⁴R⁵, or 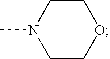
R⁴, R⁵═H, C₁₋₄ alkyl;
Y═X¹—(CH₂)ₙ—X²;
X¹═O, a direct bond;
n=1-6, provided that when X¹ is O, n=2-6;
X²═H, OH, OR³, OC(O)R³, NHC(O)R³, NHS(O)₂R³, C(O)NR⁶R⁷, OC(O)NR⁶R⁷, NHC(O)NR⁶R⁷, C(O)NR⁸R^{9,}
R⁶, R⁷═H, C₁₋₄ alkyl; and
R⁸, R⁹═H, C₁₋₄ alkyl, OH, OCH₃, provided that only one of R⁸ and R⁹ can be OH or OCH₃.

According to the methods of the present invention, a compound of formulas I, II, or III, or a pharmaceutically acceptable salt thereof, is topically administered to the eye. Examples of pharmaceutically acceptable salts of the compounds of formulas I, II, and III include, but are not limited to, inorganic acid salts such as hydrochloride, hydrobromide, sulfate and phosphate; organic acid salts such as acetate, maleate, fumarate, tartrate and citrate; alkali metal salts such as sodium salt and potassium salt; alkaline earth metal salts such as magnesium salt and calcium salt; metal salts such as aluminum salt and zinc salt; and organic amine addition salts such as triethylamine addition salt (also known as tromethamine), morpholine addition salt and piperidine addition salt.

It is recognized that compounds of Formulas (I)-(III) can contain one or more chiral centers. This invention contemplates all enantiomers, diastereomers, and mixtures thereof. In the above definitions, the total number of carbon atoms in a substituent group is indicated by the Cᵢ-Cⱼ prefix, where the numbers i and j define the number of carbon atoms; this definition includes straight chain, branched chain, and cyclic alkyl or (cyclic alkyl)alkyl groups.

Substituents identified in the formulas above may be present either singly or multiply when incorporated into the indicated structural unit.

The following examples are provided to illustrate different aspects and embodiments of the present invention. These examples are not intended in any way to limit the disclosed invention. The following abbreviations have been used in the examples: DCE—dichloroehtane, DCM—dichloromethane, EDCl 1-(3-dimethylaminopropyl)-3-ethyl-carbodiimide hydrochloride, EtOAc—ethylacetate, HOBT—hydroxybenzotriazole hydrate, MeOH—methanol, THF—tetrahydrofuran.

### EXAMPLE 1

### (S)-2-{11-[3-Dimethylamino-prop-(Z)-ylidene]-6,11-dihydro-dibenz[b,e]-oxepin-2-yl}-N-tetrahydro-furan-2-ylmethyl)acetamide fumaric acid salt

(S)-2-{11-[3-Dimethylamino-prop-(Z)-ylidene]-6,11-dihydro-dibenz[b,e]oxepin-2-yl}-N-tetrahydro-furan-2-ylmethyl)acetamide fumaric acid salt was prepared by a multiple step reaction sequence.

### {11-[3-Dimethylamino-prop-(Z)-ylidene]-6,11-dihydro-dibenz[b,e]oxepin-2-yl}-acetic acid methyl ester

Olopatadine hydrochloride [(0.25 g, 0.67 mmol)] was dissolved in methanol (10 mL) and treated with excess of acetyl chloride (0.2 g, 2.5 mmol) at 23° C. The solution was stirred for 2 hr, then poured into dilute aqueous NaHCO₃ (50 mL), extracted with ethyl acetate (2×20 mL), dried over Na₂SO₄, filtered and concentrated. The crude product was purified by flash chromatography on silica gel [eluent: methanol/DCM gradient (5%-20%)], to furnish {11-[3-Dimethylamino-prop-(Z)-ylidene]-6,11-dihydro-dibenz[b,e]oxepin-2-yl}-acetic acid methyl ester (0.22 g, 0.63 mmol) in 94% yield. Mass spectra: m/z 352 [M+H]⁺. ¹H NMR (CDCl₃, 600 MHz) δ 7.31 (m, 1H), 7.24 (m, 3H), 7.07 (d, J=2.4 Hz, 1 H), 7.05 (dd, J=8.4 Hz, 1 H), 6.80 (d, J=8.4 Hz, 1 H), 5.71 (t, J=7.2 Hz, 1H), 3.70 (s, 3H), 3.53 (s, 2H), 2.58 (dd, J=14.4, 72 Hz, 2H), 2.45 (t, J=7.2 Hz, 2H), 2.23 (s, 6H). ¹³C NMR (CDCl₃, 150 MHz) δ 172.15, 154.58, 145.60, 139.67, 133.64, 132.00, 130.81, 129.90, 129.04, 127.44, 127.43, 126.29, 125.68, 123.93, 119.74, 70.40, 59.49, 51.96, 45.40, 40.25, 28.06.

### (S)-2-{11-[3-Dimethylamino-prop-(Z)-ylidene]-6,11-dihydro-dibenz[b,e]oxepin-2-yl}-N-tetrahydro-furan-2-ylmethyl)acetamide fumaric acid salt

In a sealed tube, {11-[3-dimethylamino-prop-(Z)-ylidene]-6,11-dihydro-dibenz[b,e]oxepin-2-yl}-acetic acid methyl ester (0.22 g, 0.63 mmol) was dissolved in THF (5 mL) and (S)-tetrahydrofurfurylamine (1 g, 9.9 mmol) added. The solution was heated to 100° C. for 20.5 hr, allowed to cool to 23° C., poured into dilute aqueous NaHCO₃ (50 mL), extracted with ethyl acetate (2×20 mL), dried over Na₂SO₄, filtered and concentrated. The crude product was purified by flash chromatography on silica gel [eluent: methanol/DCM gradient (5%-10%)], to furnish (S)-2-{11-[3-dimethylamino-prop-(Z)-ylidene]-6,11-dihydro-dibenz[b,e]oxepin-2-yl}-N-tetrahydro-furan-2-ylmethyl)acetamide (0.21 g, 0.5 mmol) in 80% yield. The purified amide was dissolved in methanol and fumaric acid (0.058 g, 0.5 mmol) added. The solution was concentrated under vacuum to afford (S)-2-{11-[3-dimethylamino-prop-(Z)-ylidene]-6,11-dihydro-dibenz[b,e]oxepin-2-yl}-N-tetrahydro-furan-2-ylmethyl)acetamide fumaric acid salt. Mass spectra: m/z 421 [M+H]⁺. ¹H NMR (D₂O, 600 MHz) δ 7.45 (m, 3H), 7.37 (d, J=6.6 Hz, 1H), 7.24 (d, J=9.0 Hz, 1H), 7.20 (s, 1H), 7.01 (d, J=9.0 Hz, 1H), 6.73 (s, 2H), 5.82 (m, 1H), 4.05 (m, 1H), 3.69 (t, J=6.6 Hz, 2H), 3.59 (m, 2H), 3.33 (m, 4H), 2.86 (s, 7H), 1.95 (m, 1H), 1.82 (m, 1H), 1.78 (m, 1H), 1.49 (m, 1H).

### EXAMPLE 2

### 1-[4-(2-Diethylamino-ethyl)-piperidin-1-yl]-2-{11-[3-dimethylamino-prop-(Z)-ylidene]-6,11-dihydro-dibenz[b,e]oxepin-2-yl}-ethanone fumaric acid salt

Olopatadine hydrochloride (0.26 g, 0.7 mmol), HOBT (0.12 g, 0.9 mmol), 4-(2-diethylamino-ethyl)-piperidine (0.16 g, 0.8 mmol) and triethylamine were dissolved in THF (20 mL). EDCl (0.15 g, 0.79 mmol) was added the solution stirred for 16 hr at 23° C., poured into dilute aqueous NaHCO₃ (50 mL), extracted with ethyl acetate (2×20 mL), dried over Na₂SO₄, filtered and concentrated. The crude product was purified by flash chromatography on silica gel [eluent: methanol/DCM (20%)], to furnish 1-[4-(2-diethylamino-ethyl)-piperidin-1-yl]-2-{11-[3-dimethylamino-prop-(Z)-ylidene]-6, 11-dihydro-dibenz[b,e]oxepin-2-yl}-ethanone (0.37 g, 0.7 mmol) in 99% yield. The purified amide (0.10 g, 0.2 mmol) was dissolved in methanol (5 mL) and fumaric acid (0.023 g, 0.2 mmol) added. The solution was concentrated under vacuum to afford 1-[4-(2-diethylamino-ethyl)-piperidin-1-yl]-2-{11-[3-dimethylamino-prop-(Z)-ylidene]-6,11-dihydro-dibenz[b,e]oxepin-2-yl}-ethanone fumaric acid salt. Mass spectra: m/z 505 [M+H]⁺. ¹H NMR (D₂O, 600 MHz) δ 7.46 (m, 3H), 7.42 (m, 1H), 7.22 (d, J=7.8 Hz, 1H), 7.13 (s, 1H), 7.01 (d, J=7.8 Hz, 1H), 6.67 (s, 3H), 5.80 (t, J=8.4 Hz, 1H), 4.38 (d, J=13.2 Hz, 1H), 3.92 (m, 1H), 3.86 (d, J=15.6 Hz, 1H), 3.77 (d, J=15.6 Hz, 1H), 3.36 (t, J=7.8 Hz, 2H), 3.06 (bm, 6H), 2.74 (bs, 8H), 2.72 (t, J=10.8 Hz, 1H), 1.75 (d, J=13.2 Hz, 1H), 1.55 (m, 3H). ¹³C NMR (CDCl₃, 150 MHz) δ 175.39, 146.57, 137.85, 132.47, 131.44, 130.59, 129.29, 127.93, 123.09, 73.76, 59.84, 50.30, 50.14, 49.53, 45.63, 42.06, 35.71, 34.02, 32.12, 27.73, 11.22.

The following examples 2-6 were prepared in a manner similar to Examples 1 and 2.

### EXAMPLE 3

### 2-[11Z-(3-Dimethylaminopropylidene)-6,11-dihydrodibenzo[b,e]oxepin-2-yl]acetamide

Mass spectra: m/z 337 [M+H]⁺. ¹H NMR (DMSO-d₆, 300 MHz) δ 7.35 (m, 1H), 7.21 (m, 3H), 7.05 (m, 2H), 6.75 (d, 1H), 5.78 (t, 1H), 3.30 (s, 2H), 2.40 (m, 6H), 2.09 (s, 6H). ¹³C NMR (DMSO-d₆, 75 MHz) δ 172.75, 153.94, 145.62, 139.15, 133.98, 131.95, 131.46, 130.22, 129.41, 128.78, 128.01, 127.78, 126.23, 119.30, 59.06, 45.32, 41.57, 40.80, 40.52, 40.25, 39.69, 39.41, 39.17, 27.77.

### EXAMPLE 4

### 2-[11Z-(3-Dimethylaminopropylidene)-6,11-dihydrodibenzo[b,e]oxepin-2-yl]-N-methylacetamide

Mass spectra: m/z 351 [M+H]⁺. ¹H NMR (DMSO-d₆, 300 MHz) δ 7.30 (m, 4H), 7.21 (m, 3H), 7.10 (m, 2H), 6.75 (d, 1H), 5.65 (t, 1H), 3.53 (s, 2H), 2.58 (m, 4H), 2.38 (m, 2H), 2.15 (s, 6H). ¹³C NMR (DMSO-d₆, 75 MHz) 171.05, 154.00, 145.58, 139.16, 133.96, 131.84, 131.44, 130.15, 129.40, 128.70, 128.00, 127.78, 126.24, 123.77, 119.36, 69.91,59.06, 45.28, 41.73, 40.81, 40.53, 40.26, 39.98, 39.70, 39.42, 39.14, 27.77, 25.95.

### EXAMPLE 5

### 2-[11Z-(3-Dimethylaminopropylidene)-6,11-dihydrodibenzo[b,e]oxepin-2-yl]-N-(2-hydroxyethyl)acetamide

Mass spectra: m/z 381 [M+H]⁺. ¹H NMR (DMSO-d₆, 300 MHz) δ 7.99 (bs, 1H), 7.25 (m, 4H), 7.10 (m, 2H), 6.75 (d, 1H), 3.45 (m, 2H), 3.30 (s,2H), 3.1 (m, 2H) 2.40 (m, 6H), 2.15 (s, 6H). ¹³C NMR (DMSO-d₆, 75 MHz) 170.77, 153.97, 145.61, 139.15, 133.96, 131.89, 131.44, 130.17, 129.41, 128.75, 128.01, 127.78, 126.23, 123.72, 119.33, 69.89, 60.29, 59.04, 45.28, 41.98, 41.69, 40.80, 40.52, 40.25, 39.97, 39.69, 39.41, 39.13, 27.76.

### EXAMPLE 6

### 2-[11Z-(3-Dimethylaminopropylidene)-6,11-dihydrodibenzo[b,e]oxepin-2-yl]-N,N-dimethylacetamide

Mass spectra: m/z 365 [M+H]⁺. ¹H NMR (DMSO-d₆, 300 MHz) 6 7.40 (m, 4H), 6.95 (m, 2H), 6.75 (d, 1H), 5.70 (t, 1H), 3.60 (s, 2H), 2.95 (s, 3H), 2.75 (s, 3H) 2.45 (m, 4H), 2.38 (m, 2H), 2.05 (s, 6H). ¹³C NMR (DMSO-d₆, 75 MHz) 172.75, 153.94, 145.62, 139.15, 133.96, 131.46, 130.22, 129.41, 128.78, 128.01, 127.78, 126.23, 123.73, 119.30, 69.89, 59.06, 45.32, 41.57, 40.80, 40.52, 40.25, 39.69, 39.41, 39.13, 27.77.

### EXAMPLE 7

### {3-[2-{2-[4-(2-Diethylamino-ethyl)-piperidin-1-yl]-ethyl}-6H-6,11-dibenz[b,e]oxepin-(11Z)-ylidene]-propyl}-dimethylamine fumaric acid salt

1-[4-(2-Diethylamino-ethyl)-piperidin-1-yl]-2-{11-[3-dimethylamino-prop-(Z)-ylidene]-6,11-dihydro-dibenz[b,e]oxepin-2-yl}-ethanone (0.37 g, 0.74 mmol) was dissolved in ether (20 mL), the solution cooled to 0° C. with an ice bath and LAH/THF (1M, 3.0 mL, 3.0 mmol) added. The solution was heated to reflux for 17 hr, allowed to cool to 23° C., quenched by careful addition of methanol (1 mL), 50% aq. NaOH (0.1 mL), and ether (5 mL), filtered and concentrated. The crude product was purified by flash chromatography on silica gel [eluent: methanol/DCM gradient (20%-20% with 2% TEA)], to furnish amine (0.21 g, 0.43 mmol) in 58% yield. The purified amine was dissolved in methanol and fumaric acid (0.05 g, 0.43 mmol) added. The solution was concentrated under vacuum to afford {3-[2-{2-[4-(2-diethylamino-ethyl)-piperidin-1-yl]-ethyl}-6H-6,11-dibenz[b,e]oxepin-(11Z)-ylidene]-propyl}-dimethylamine fumaric acid salt (AL-43437A): mp=65-67° C. Mass spectra: m/z490 [M+H]⁺. ¹H NMR (D₂O, 600 MHz) δ 7.38 (m, 3H), 7.31 (m, 1H), 7.17 (dd, J=8.4, 2.0 Hz, 1H), 7.11 (d, J=2.0 Hz, 1H), 6.94 (d, J=8.4 Hz, 1H), 6.46 (s, 1H), 5.75 (t, J=7.2 Hz, 1H), 5.3 (bm, 2H), 3.56 (d, J=12.0 Hz, 2H), 3.3-3.2 (m, 4H), 3.12 (q, J=7.2 Hz, 4H), 3.1 (m, 2H), 2.9 (m, 4H), 2.8-2.7 (m, 8H), 1.95 (d, J=12.0 Hz, 2H), 1.66 (m, 3H), 1.4 (m, 2H), 1.23 (t, J=7.2 Hz, 6H). ¹³C NMR (CDCl₃, 150 MHz) δ 174.35, 153.71, 141.70, 135.31, 133.20, 130.36, 129.44, 128.44, 127.51, 126.38, 125.38, 120.34, 70.84, 56.80, 47.29, 28.28, 24.75, 8.17. Combustion analysis calcd for C₃₂H₄₇N₃O.1.2 C₄H₄O₄.4 H₂O: C, 63.04; H, 8.60; N, 5.99. found: C, 63.36; H, 8.24; N, 6.05.

The following example was prepared in a manner similar to Example 7

### EXAMPLE 8

### {3-[2-(2-Aminoethyl)-6H-dibenzo[b,e]oxepin-11-ylidene]propyl}-dimethylamine

Mass spectra: m/z 322 [M+H]⁺. ¹H NMR (CD₃OD, 300 MHz) δ 7.3 (m, 4H), 7.00 (m, 2H), 6.75 (d, 1H), 5.65 (t, 1H), 5.3 (bm, 2H), 3.3 (s, 2H), 2.81 (m, 2H), 2.65 (m, 2H), 2.60 (m, 2H),2.50 (m, 2H) 2.25 (s, 6H). ¹³C NMR (CD₃OD, 75 MHz) δ 155.89, 147.07, 142.50, 135.76, 133.24, 132.73, 131.25, 131.10, 130.45, 129.03, 128.84, 125.50, 126.00, 121.25, 71.88, 60.65, 50.27, 49.44, 49.71, 49.42, 49.14, 48.85, 48.57, 45.73, 44.63, 39.35, 29.20.

### EXAMPLE 9

### N-{2-[11-(3-Dimethylaminoprop-(Z)-ylidene)-6,11-dihydrodibenzo[b,e]-oxepin-2-yl]ethyl}- methanesulfonamide

To a solution of {3-[2-(2-aminoethyl)-6H-dibenzo[b,e]oxepin-11-ylidene]propyl}dimethylamine (85 mg, 0.26 mmol) in DCM (3 mL) at room temperature was added triethylamine (50 μL, 0.39 mmol). The resulting mixture was cooled to 0° C. and treated drop wise with methanesulfonylchloride (20 μL, 0.29 mmol). The resulting mixture was stirred at 020 C. for 15 minutes when deemed complete by TLC analysis [EtOAc/MeOH (1:1)].

The reaction mixture was concentrated and the residue was partitioned between water (20 mL) and EtOAc (15 mL). The aqueous phase was further extracted with EtOAc (3×15 mL). The combined organic extracts were washed with saturated aqueous NaCl (20 mL) and then dried over MgSO₄ and concentrated to an orange solid. This material was subjected to flash column chromatography eluting with EtOAc to EtOAc/MeOH (1:1) to provide N-{2-[11-(3-dimethylaminopropylidene)-6,11-dihydrodibenzo[b,e]-oxepin-2-yl]ethyl}methanesulfonamide (42 mg, 40%) as a yellow solid. HPLC analysis showed 90.1% (AUC).

Mass spectra: m/z 401 [M+H]⁺. ¹H NMR (CD₃OD, 300 MHz) δ 7.40 (m, 4H), 7.10 (m, 2H), 6.75 (d, 1H), 5.70 (t, 1H), 5.20 (bs, 1H), 4.85 (s, 1H) 3.30 (m, 2H), 2.95 (d, 2H), 2.65 (m, 4H), 2.30 (s, 6H) ¹³C NMR (CD₃OD, 75 MHz) δ 142.63, 135.71, 133.01, 131.35, 130.69, 130.50, 129.09, 128.88, 127.52, 121.26, 71.86, 60.44, 50.29, 50.01, 49.72, 49.44, 49.16, 48.87, 48.59, 46.20, 45.59, 46.20, 45.59. 40.35, 37.15, 29.00.

The following example was prepared in a manner similar to Example 9.

### EXAMPLE 10

### N-[2-[11-(3-Dimethylaminoprop-(Z)-ylidene)-6,11-dihydrodibenzo[b,e]-oxepin-2-yl]ethyl}-acetamide

Mass spectra: m/z 366 [M+H]⁺. ¹H NMR (CD₃OD, 300 MHz) δ 7.30 (m, 4H), 7.05 (m, 2H), 6.75 (d, 1H), 5.75 (t, 1H), 5.20 (bs, 1H), 4.90 (s, 1H) 3.40 (m, 2H), 2.95 (m, 2H), 2.65 (m, 2H), 2.55 (m, 2H) 2.25 (s, 6H), 1.85 (s, 3H) ¹³C NMR (CD₃OD, 75 MHz) δ 173.56, 155.92, 147.12, 142.48, 135.74, 131.29, 131.13, 130.48, 129.03, 128.86, 127.50, 121.18, 71.84, 60.66, 50.30, 50.01, 49.73, 49.45, 49.16, 48.88, 48.60, 45.79, 42.68, 35.97, 29.20, 23.01.

### EXAMPLE 11

### [11-(1-Methylpiperidin-4-ylidene)-6,11-dihydrodibenzo[b,e]oxepin-2-yloxy]acetic acid hydrochloride

[11-(1-Methylpiperidin-4-ylidene)-6,11-dihydrodibenzo[b,e]oxepin-2-yloxy]-acetic acid hydrochloride was prepared by the multiple step reaction sequence outline below.

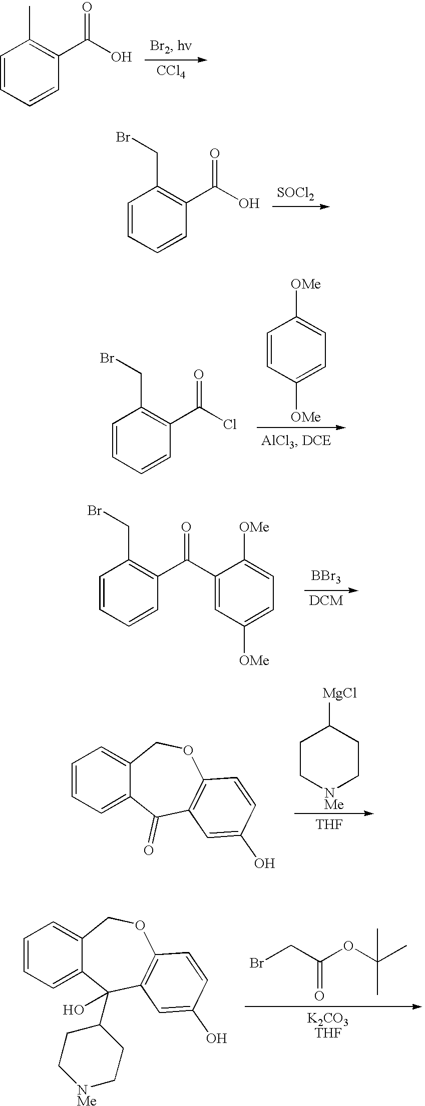

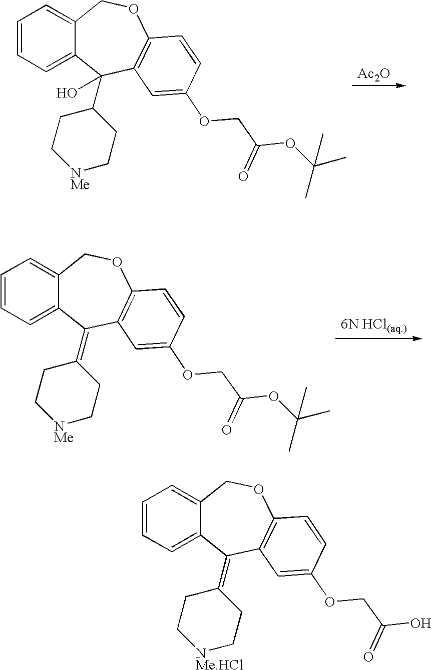

### 2-Bromomethylbenzoic acid

A solution of o-Toluic acid (15.0 g, 110.3 mmol) in CCl₄ (150 mL) was warmed to reflux and treated with a solution of Br₂ (17.6 g, 110.3 mmol) in CCl₄ (150 mL) whilst irradiating the solution with a 200 W tungsten lamp. Bromine addition was such that a pale orange color was observed throughout the addition. A vigorous reflux and evolution of hydrogen bromide was also observed during addition.

At the end of the addition a colorless suspension was observed and heating and irradiation were continued for a further 5 minutes before being removed. TLC analysis [hexane/EtOAc (1:1)] of the mixture indicated that no starting material remained. The solution was cooled to 65° C. and treated with n-hexane then further cooled to room temperature. The precipitated solid was removed by filtration and washed with n-hexane and dried in a vacuum oven at 40° C. (−30 inHg). This provided the desired 2-Bromomethylbenzoic acid as a colorless solid (20. 0 g, 84%), which was deemed pure enough (NMR analysis) for use in subsequent steps without the need for further purification.

### 2-Bromomethylbenzoyl chloride

2-Bromomethylbenzoic acid (20.0 g, 93.0 mmol) was dissolved in thionyl chloride (110.7 g, 68 mL, 930.0 mmol) and heated at reflux for 3 hours when deemed complete by GC/MS analysis (derivatized to the methyl ester by adding an aliquot of the reaction mixture to excess methanol and heating at reflux for 5 minutes). Excess thionyl chloride was removed by vacuum distillation and the crude product was further dried under vacuum (10 mmHg) to give the desired 2-bromomethylbenzoyl chloride as a pale yellow solid (21.67 g, 100%), which was used crude in subsequent manipulations.

### (2-Bromomethylphenyl)-(2,5-dimethoxyphenyl)methanone

A solution of 2-bromomethylbenzoyl chloride (21.67 g, 93.0 mmol) in 1,2-dichloroethane (DCE) (900 mL) was added drop wise to a suspension of AlCl₃ (12.4 g, 93.0 mmol) in DCE (900 mL) at 0° C. A slight exotherm (5° C.) was observed and internal temperature was maintained in the range 0-5° C. throughout the addition. The resulting pale yellow solution was stirred at 0° C. for 15 minutes and then treated drop wise with 1,4-dimethoxybenzene (12.83 g, 93.0 mmol), again maintaining 0-5° C. via rate of addition. During the addition the solution became dark green in color and the resulting mixture was allowed to warm to 15° C. over a 12 hour period. TLC analysis [hexane/EtOAc (4:1)] showed the reaction was complete.

The reaction mixture was poured onto ice-water (1.8 L) and the organic phase was separated. The aqueous phase was further extracted with DCM (3×1.0 L) and the combined organics were dried over Na₂SO₄ and concentrated to approximately 1.0 L of residual volume. This solution of (2-Bromomethylphenyl)-(2,5-dimethoxyphenyl)methanone was used crude in the next step. [Note: the crude material appeared to decompose on standing at room temperature and was found to be unstable to silica gel chromatography.]

### (2-Bromomethylphenyl)-(2,5-dihydroxyphenyl)methanone

(2-Bromomethylphenyl)-(2,5-dimethoxyphenyl)methanone [31.2 g, 93.0 mmol (assume 100% from last step)] in DCE (1.0 L) was diluted with DCM (500 mL) and cooled to 0° C. The dark green solution was treated drop wise with boron tribromide (66.0 g, 26.0 mL, 0.26 mol, 2.8 equivalents), whilst maintaining 0-5° C. (adequate venting is required and the reaction is exothermic). During the addition, the solution became dark red in color and this solution was stirred and warmed to room temperature over 3 hours. At this point TLC analysis [hexane/EtOAc (4:1)] showed the reaction was complete.

The reaction mixture was carefully quenched by addition of methanol (˜10 mL or until reaction ceases) and then concentrated to a red solid. The solid was partitioned between water (1.0 L) and DCM (1.0 L). The aqueous phase was further extracted with DCM (2×1.0 L) and the combined organic extracts were dried over Na₂SO₄ (note: discoloration from dark red to dark brown was observed when MgSO₄ was used as a drying agent) and concentrated to a red oil (27.8 g, 97%) which was used without further purification in subsequent manipulations. [Note: the crude material appeared to decompose on standing at room temperature and was found to be unstable to silica gel chromatography.]

### 2-Hydroxy-6H-dibenzo[b,e]oxepin-11-one

A solution of (2-bromomethylphenyl)-(2,5-dihydroxyphenyl)methanone (27.8 g, 90.5 mmol) in MeCN (2.0 L) was treated with finely powdered K₂CO₃ (25.0 g, 181.0 mmol) and the resulting suspension was stirred at room temperature for 18 hours when deemed complete by TLC analysis [hexane/EtOAc (4:1)].

The reaction mixture was diluted with water (1.0 L) and acidified to pH ˜3-4 by addition of 1N aqueous HCl. This mixture was then extracted with EtOAc (3×1.5 L) and the combined organic extracts were dried over Na₂SO₄ and concentrated to a brown solid. This solid was subjected to flash column chromatography eluting with hexane/EtOAc (2:1) to provide 2-Hydroxy-6H-dibenzo[b,e]oxepin-11-one (9.2 g, 35%) as a yellow solid.

### 11-(1-Methylpiperidin-4-yl)-6,11-dihydrodibenzo[b,e]oxepine-2-11-diol

2-Hydroxy-6H-dibenzo[b,e]oxepin-11-one (1.0 g, 4.42 mmol) in THF (30 mL) was treated at 0° C. with a solution of 1-methypiperidine-4-magnesium chloride* (0.79 M, 11.8 mL, 9.3 mmol). The resulting orange solution was stirred at 0° C. for 1 hour when deemed complete by TLC analysis [hexane/EtOAc (1:1)].
[The Grignard solution was prepared from N-methyl-4-chloropiperidine (1.0 equivalent, free base only) and fresh Mg turnings (1.1 equivalents) in THF (1 mL/g). This mixture was treated with an iodine crystal and heated at reflux for 12-24 hours when a milky suspension had formed.]

The reaction mixture was quenched by addition of saturated aqueous NH₄Cl (10 mL) and partitioned between EtOAc (50 mL) and water (30 mL). The aqueous phase was further extracted with EtOAc (3×50 mL) and the combined organic extracts were dried over Na₂SO₄ and concentrated to an orange oil. This oil was triturated with EtOAc to provide 11-(1-Methylpiperidin-4-yl)-6,11-dihydrodibenzo[b,e]oxepine-2-11-diol (715 mg, 50%) as a brown solid, which was homogenous by TLC and NMR analysis.

### [11-Hydroxy-11-(1-methylpiperidin-4-yl)-6,11-dihydrodibenzo[b,e]oxepin-2-yloxy]acetic acid tert-butyl ester

11-(1-Methylpiperidin-4-yl)-6,11-dihydrodibenzo[b,e]oxepine-2-11-diol (563 mg, 1.73 mmol) in DMF (6 mL) was treated with finely powdered K₂CO₃ (478 mg, 3.46 mmol) followed by tert-butylbromoacetate (372 mg, 282 μL, 1.91 mmol) and the resulting mixture was stirred at room temperature for 14 hours when deemed complete by TLC analysis [DCM/MeOH (92:8)].

Saturated aqueous NaCl (3 mL) was added resulting in formation of an off-white precipitate. This was collected by filtration and washed with water and n-heptane, then dried in a vacuum oven at 30° C. (−30 inHg) to give [11-hydroxy-11-(1-methylpiperidin-4-yl)-6,11-dihydrodibenzo[b,e]oxepin-2-yloxy]-acetic acid tert-butyl ester (765 mg, 101%) as an off-white foam. NMR analysis showed a single impurity (10-15% total) which was found to be impossible to remove either by recrystallization or column chromatography.

### [11-(1-Methylpiperidin-4-ylidene)-6,11-dihydrodibenzo[b,e]oxepin-2-yloxy]acetic acid tert-butyl ester

[11-Hydroxy-11-(1-methylpiperidin-4-yl)-6,11-dihydrodibenzo[b,e]oxepin-2-yloxy]acetic acid tert-butyl ester (760 mg, 1.73 mmol) was dissolved in acetic anhydride (4 mL) and the resulting orange solution was stirred at 80° C. for 48 hours when deemed complete by TLC analysis [DCM/MeOH (8:2)].

Volatiles were removed in vacuo and the resulting residue was subjected to flash column chromatography eluting with DCM/MeOH (97:3) to provide [11-(1-methylpiperidin-4-ylidene)-6,11-dihydrodibenzo[b,e]oxepin-2-yloxy]-acetic acid tert-butyl ester (425 mg, 58%) as an orange oil.

### [11-(1-Methylpiperidin4-ylidene)-6,11-dihydrodibenzo[b,e]oxepin-2-yloxy]acetic acid hydrochloride

[11-(1-Methylpiperidin-4-ylidene)-6,11-dihydrodibenzo[b,e]oxepin-2-yloxy]-acetic acid tert-butyl ester (310 mg, 0.736 mmol) was dissolved in 6N aqueous hydrochloric acid (5 mL) and heated at 50° C. for 1 hour when TLC analysis [DCM/MeOH (97:3)] indicated no starting material remained.

The reaction mixture was concentrated to a brown solid.

The solid was recrystallized from methanol/acetone to give [11-(1-methylpiperidin-4-ylidene)-6,11-dihydrodibenzo[b,e]oxepin-2-yloxy]acetic acid hydrochloride (96 mg, 33%) as a tan solid. Mass spectra: m/z 366 [M+H]⁺. ¹H NMR (DMSO-d₆, 300 MHz) δ 7.40 (m, 3H), 7.15 (m, 1H), 6.65 (m, 2H), 6.50 (s, 1H) 5.15 (dd, 2H), 4.35 (d, 2H), 3.7 (m, 2H), 3.35 (s, 2H),3.20 (s, 2H) 2.80 (m, 2H), 2.60 (m, 2H),2.50 (s, 3H) ¹³C NMR (DMSO-d₆, 75 MHz) δ 166.38, 166.12, 150.87, 147.25, 141.91, 133.96, 133.82, 133.75, 128.97, 128.86, 128.61, 128.44, 128.11, 127.12, 127.04, 124.91, 124.83, 120.14, 116.98, 116.68, 116.64, 68.94, 63.32, 61.04, 60.93, 60.70, 60.43, 58.41, 55.26, 50.11, 45.42, 24.91, 24.77, 24.86

### Synthesis of N-{2-[11-(3-Dimethylaminopropylidene)-6,11-dihydrodibenzo[b,e]oxepin-2-yl]ethyl}methanesulfonamide

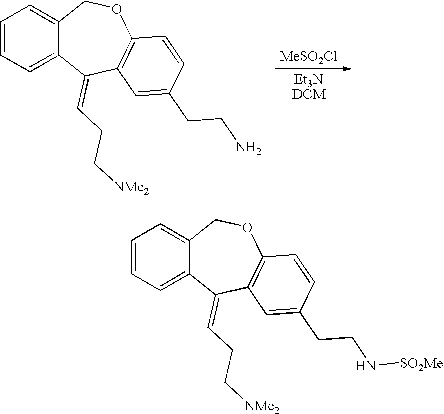

To a solution of {3-[2-(2-Aminoethyl)-6H-dibenzo[b,e]oxepin-11-ylidene]propyl}dimethylamine (85 mg, 0.26 mmol) in DCM (3 mL) at room temperature was added triethylamine (50 μL, 0.39 mmol). The resulting mixture was cooled to 0° C. and treated drop wise with methanesulfonylchloride (20 μL, 0.29 mmol). The resulting mixture was stirred at 0° C. for 15 minutes when deemed complete by TLC analysis [EtOAc/MeOH (1:1)].

The reaction mixture was concentrated and the residue was partitioned between water (20 mL) and EtOAc (15 mL). The aqueous phase was further extracted with EtOAc (3×15 mL). The combined organic extracts were washed with saturated aqueous NaCl (20 mL) and then dried over MgSO₄ and concentrated to an orange solid. This material was subjected to flash column chromatography eluting with EtOAc to EtOAc/MeOH (1:1) to provide N-{2-[11-(3-Dimethylaminopropylidene)-6,11-dihydrodibenzo[b,e]-oxepin-2-yl]ethyl}methanesulfonamide (42 mg, 40%) as a yellow solid. HPLC analysis showed 90.1% (AUC).

Mass spectra: m/z 401 [M+H]⁺. ¹H NMR (CD₃OD, 300 MHz) δ 7.40 (m, 4H), 7.10 (m, 2H), 6.75 (d, 1H), 5.70 (t, 1H), 5.20 (bs, 1H), 4.85 (s, 1H) 3.30 (m, 2H), 2.95 (d, 2H), 2.65 (m, 4H), 2.30 (s, 6H) ¹³C NMR (CD₃OD, 75 MHz) δ 142.63, 135.71, 133.01, 131.35, 130.69, 130.50, 129.09, 128.88, 127.52, 121.26, 71.86, 60.44, 50.29, 50.01, 49.72, 49.44, 49.16, 48.87, 48.59, 46.20, 45.59, 46.20, 45.59. 40.35, 37.15, 29.00.

### Synthesis of N-{2-[11-(3-Dimethylaminopropylidene)-6,11-dihydrodibenzo[b,e]oxepin-2-yl]ethyl}methanesulfonamide

Mass spectra: m/z 366 [M+H]⁺. ¹H NMR (CD₃OD, 300 MHz) δ 7.30 (m, 4H), 7.05 (m, 2H), 6.75 (d, 1H), 5.75 (t, 1H), 5.20 (bs, 1H), 4.90 (s, 1H) 3.40 (m, 2H), 2.95 (m, 2H), 2.65 (m, 2H), 2.55 (m, 2H) 2.25 (s, 6H), 1.85 (s, 3H) ¹³C NMR (CD₃OD, 75 MHz) δ 173.56, 155.92, 147.12, 142.48, 135.74, 131.29, 131.13, 130.48, 129.03, 128.86, 127.50, 121.18, 71.84, 60.66, 50.30, 50.01, 49.73, 49.45, 49.16, 48.88, 48.60, 45.79, 42.68, 35.97, 29.20, 23.01.

### EXAMPLE 12

### Radioligand Binding Studies and Histamine Studies

Histamine receptor binding was conducted in washed rodent brain homogenates. Briefly, guinea pig forebrains (for H1 and H2 receptors) and rat forebrains (for H3 receptor), obtained from Pel-Freez (Rogers, Ark.) were homogenized in 20 ml ice-cold phosphate buffered saline (PBS; pH 7.4) using a Polytron tissue disrupter (setting 5-7 for 10 sec) and centrifuged at 40,000 g for 15 min on a Beckman J2-MC centrifuge. The supernatants were discarded, the tissue pellets re-homogenized in fresh PBS buffer and centrifuged as described above. The final pellets were dispersed in 50 mM sodium potassium phosphate buffer (pH 7.5) and kept frozen at −40° C. until used in the binding assays.

The H1 histamine receptor binding assays were performed according to previously published methods with minor modifications (Chang, R. S. L., Tran, V. T., and Snyder, S. H. J. Neurochem., 32:1653-1663, 1979, Hill, S. J., Young, J. M., and Marrian, D. H. Nature, 270:361-363, 1977, Gajtkowski, G. A., Norris, D. B., Rising, T. J., and Wood, T. P. Nature, 304:65-67,1983, Korte, A., Myers, J., Shih, N.-Y., Egan, R. W., and Clark, M. A. Biochem. Biophys. Res. Comm., 168:979-986, 1990 . Briefly, 50 μl of [3H]pyrilamine (1-2 nM); 24 Ci/mmol from NEN, Boston, Mass.), [3H]tiotidine (4-5 nM; 87 Ci/mmol from NEN, Boston, Mass.) or [3H]N-methyl histamine (1-2 nM; 84 Ci/mmol from NEN, Boston, Mass.) were incubated with 200 μl of 2.5-4.0 mg/ml of frozen-thawed brain homogenates in the presence or absence of unlabeled test compounds (50 μl; 10 pM-100 μM) in a total volume of 500 μl in polypropylene tubes. The non-specific binding was determined with 5 mM histamine. Drug dilutions and dispensing of assay components were performed using a computer-controlled automated system (Biomek; Beckman, Fullerton, Calif.). The assays were conducted at 23° C. for 40 min and then terminated by rapid filtration over Whatman GF/B glass fiber filters presoaked in 0.3% polyethyleneimine using a Tomtec cell harvester (Gaithersburg, Md.). The assay tubes were rinsed with 2×6 ml of ice-cold 50 mM TrisHCl buffer (pH 7.4). The filter-bound radioactivity was determined on a Wallac Beta-plate (Gaithersburg, Md.) solid scintillation counter at 40-50% efficiency. The competition binding data were analyzed using an iterative curve-fitting computer program as previously described Michel, A. D. and Whiting, R. L. Brit. J. Pharmacol., 83:460p, 1984, Sharif, N. A., Wong, E. H. F., Loury, D., Stefanich, E., Eglen, R. M., Michel, A. D., and Whiting, R. L. Brit. J. Pharmacol., 102:919-925, 1991,. The drug affinities (dissociation constants, Kis) were calculated according to the Cheng-Prussof equation (Cheng, Y.-C. and Prusoff, W. H. Biochem. Pharmacol., 22:3099-3018, 1973 ):
Ki=IC50/(1+L/Kd)
where IC50 is the concentration of the drug needed to produce 50% inhibition of the receptor binding, L is the radioligand concentration in the assay and Kd is the dissociation constant of the radioligand.

### Histamine Induced Vascular Permeability in Guinea Pig Conjunctiva

Male Dunkin Hartley Viral Antibody Free out bread guinea pigs (Charles River Labs, Portage, Mich.), 250-350 grams, (6/group) were injected intravenously (i.v.) via the marginal ear vein with 1.0 ml of Evans Blue dye (1.0 mg/ml). Forty-five (45) min post dye injection, 20 μl of test compound or vehicle was applied topically onto one eye of each experimental animal. Thirty (30) min following topical drug application, guinea pigs were anesthetized and challenged subconjunctivally with histamine (300 ng/10 μl). Responses were quantitated as previously described by Yanni, J. M., Weimer, L. K., Glaser, R. K., Lang, L. S., Robertson, S. M., and Spellman, J. M. Int. Arch. Allergy Immunol. 101:102-106, 1993

### Histamine Release from Human cConjunctival Mast Cells Preparation of Cell Suspension

Methods detailing preparation of monodispersed human conjunctival tissue mast cell and mediator release studies with these cells have been described (Miller et al. 1996). Briefly, human conjunctival tissue mast cells were isolated from post-mortem tissue donors obtained within 8 hours of death by various eye banks and transported in Optisol® corneal preservation medium. Tissues were enzymatically digested by repeated exposure (30 min. at 37° C.) to collagenase and hyaluronidase (2× with 200 U each/gm tissue, then 2-4× with 2000 U each/gm tissue) in Tyrode's buffer containing 0.1% gelatin. Each digestion mixture was filtered over Nitex® cloth (100 μm mesh) and washed with an equal volume of buffer. Filtrates were centrifuged at 825×g (7 min). Pellets were resuspended in buffer then combined for enrichment over a 1.058 g/L Percoll® cushion. The enriched pellet was washed and resuspended in supplemented RPMI 1640 medium for equilibration at 37° C.

Cells were harvested from the culture plate and counted for viability (trypan blue exclusion) and mast cell number (toluidine blue 0). Mast cells (5000/tube; 1 mL final volume) were challenged (37° C.) for 15 min with goat-anti-human IgE (10 μg/mL) following treatment (1 or 15 minutes; 37° C.) with test drug or Tyrode's buffer. Total and non-specific release controls were exposed to 0.1% Triton X-100 and goat IgG (10 μg/mL), respectively. The reaction was terminated by centrifugation (500×g, 4° C., 10 min). Supernatants were stored at −20° C. until analyzed for histamine content by R I A Miller, S. et al., Ocular Immunology and Inflammation 4(1): 39-49 (1996).

The results are reported in Table 1 below.

TABLE 1

| | | | Histamine Induced | |
|---|---|---|---|---|
| | | | Vascular | |
| | | | Permeability | Human |
| | | H. Receptor | ED50 or | Conjunctiva Mast |
| Example | | Binding | [Concentration] | Cell Histamine |
| Number | Structure | IC₅₀ (nM) | and (% inhibition) | Release IC₅₀ (uM) |
| 1 | 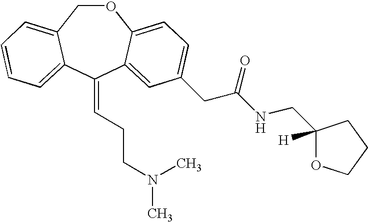 | | | 61 |
| 2 | 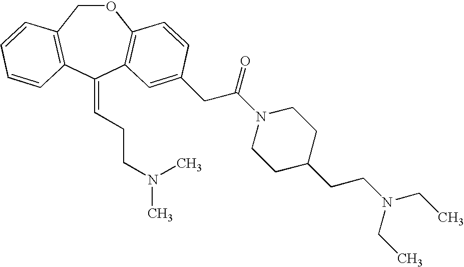 | | | 2 |
| 3 | 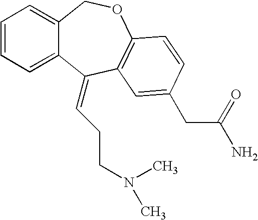 | 2.2 | 0.018 (59%) | 480 |
| 4 | 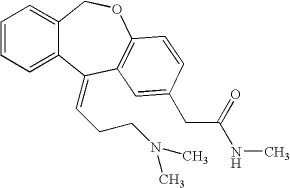 | 2.6 | 0.1% (55%) | 220 |
| 5 | 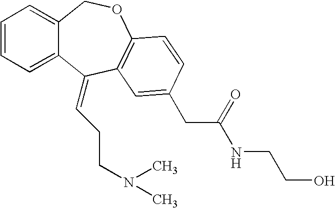 | 1.3 | 0.01% (43%) | 770 |
| 6 | 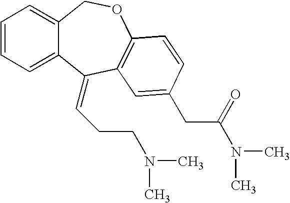 | 2.6 | 0.01% (56%) | 400 |
| 7 | 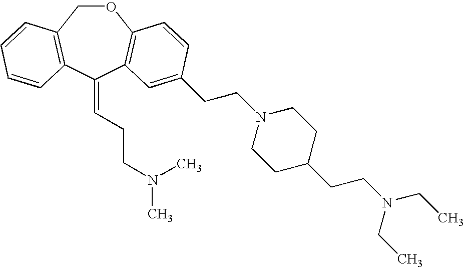 | | | |
| 8 | 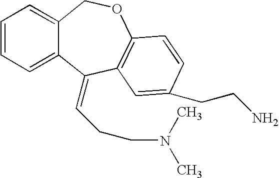 | | 0.1% (32%) | 2 |
| 9 | 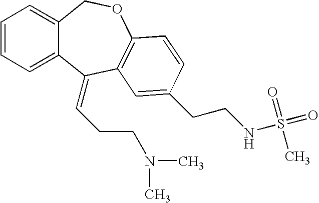 | 127 | 0.018% (74%) | 15 |
| 10 | 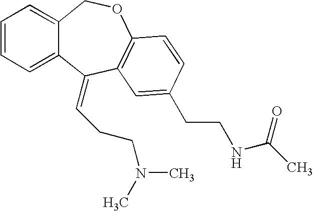 | 54 | 0.1% (41%) | 13 |
| 11 | 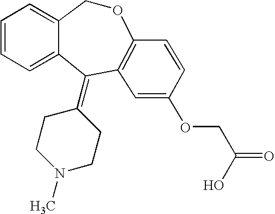 | | | 220 |

The tricyclic compounds of the present invention may be administered topically (i.e., local, organ-specific delivery) by means of conventional topical formulations, such as solutions, suspensions or gels for the eye and ear; nasal sprays or mists for the nose. The concentration of the tricyclic compound of formula I, II, or III in the formulations of the present invention will depend on the selected route of administration and dosage form, but will generally range from 0.00001 to 5 wt. %, preferably from 0.001 to 5 wt. %. For solutions intended for topical administration to the eye, the concentration of the tricyclic compound of formulas I, II, or III is preferably 0.0001 to 0.2 wt. %, and most preferably 0.01 to 0.2 wt. %. The topical compositions of the present invention are prepared according to conventional techniques and contain conventional excipients in addition to one or more tricyclic compounds of formulas I, II, or III. A general method of preparing eye drop compositions is described below:

One or more tricyclic compounds of formula I, II, or III and a tonicity-adjusting agent are added to sterilized purified water and if desired or required, one or more excipients. The tonicity-adjusting agent is present in an amount sufficient to cause the final composition to have an ophthalmically acceptable osmolality (generally about 150-450 mOsm, preferably 250-350 mOsm). Conventional excipients include preservatives, buffering agents, chelating agents or stabilizers, viscosity-enhancing agents and others. The chosen ingredients are mixed until homogeneous. After the solution is mixed, pH is adjusted (typically with NaOH or HCl) to be within a range suitable for topical ophthalmic use, preferably within the range of 4.5 to 8.

Many ophthalmically acceptable excipients are known, including, for example, sodium chloride, mannitol, glycerin or the like as a tonicity-adjusting agent; benzalkonium chloride, polyquaternium-1 or the like as a preservative; sodium hydrogenphosphate, sodium dihydrogenphosphate, boric acid or the like as a buffering agent; edetate disodium or the like as a chelating agent or stabilizer; polyvinyl alcohol, polyvinyl pyrrolidone, polyacrylic acid, polysaccharide or the like as a viscosity-enhancing agent; and sodium hydroxide, hydrochloric acid or the like as a pH controller.

According to the present invention, the tricyclic compounds of formulas I, II, and III are useful for treating ophthalmic allergic disorders, including allergic conjunctivitis, vernal conjunctivitis, vernal keratoconjunctivitis, and giant papillary conjunctivitis; nasal allergic disorders, including allergic rhinitis and sinusitis; and otic allergic disorders, including eustachian tube itching.

The eye drops produced by the above method typically need only be applied to the eyes a few times a day in an amount of one to several drops at a time, though in more severe cases the drops may be applied several times a day. A typical drop is about 30 μl.

Certain embodiments of the invention are illustrated in the following examples.

### EXAMPLE 1

### Topical Ophthalmic Solution Formulation

| Ingredient | Concentration (w/v %) |
|---|---|
| Tricyclic compound of formula I, II, or III | 0.1 |
| Dibasic Sodium Phosphate (Anhydrous) | 0.5 |
| Sodium Chloride | 0.65 |
| Benzalkonium Chloride | 0.01 |
| Sodium Hydroxide | q.s. pH 7.0 ± 0.2 |
| Hydrochloric Acid | q.s. pH 7.0 ± 0.2 |
| Purified Water | q.s. 100 |

### EXAMPLE 2

### Topical Ophthalmic Gel Formulation

| Ingredient | Concentration (w/v %) |
|---|---|
| Tricyclic compound of formula I, II, or III | 0.1 |
| Carbopol 974 P | 0.8 |
| Disodium EDTA | 0.01 |
| Polysorbate 80 | 0.05 |
| Benzalkonium Chloride, Solution | 0.01 + 5 xs |
| Sodium Hydroxide | q.s. pH 7.0 ± 0.2 |
| Hydrochloric acid | q.s. pH 7.0 ± 0.2 |
| Water for Injection | q.s. 100 |

1. A method for treating an allergic disease of the eye, ear or nose in a patient comprising topically administering to the patient a composition comprising a pharmaceutically acceptable carrier and a pharmaceutically effective amount of tricyclic compound of formula I, II, or III or a pharmaceutically acceptable salt thereof: 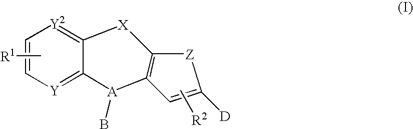
wherein:
Y, Y²═CH;
X═—CH₂—O—;
Z=-CH═CH—;
R¹, R²═Cl, Br, F, CF₃, C₁₋₆ alkyl, C₁₋₆ alkylO—;
A=C, CH, N;
provided that when A=CH or N, then B═CH₂—CH₂—CH₂—Y³, and when A=C, then B═CH—CH₂CH₂—Y³ or 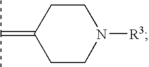
R³═C₁₋₄ alkyl;
Y³═NR⁴R⁵, or 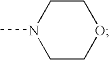
R⁴, R⁵═H, C₁₋₄ alkyl;
D=X¹—(CH₂)ₙ—X²;
X¹=a direct bond;
n=1-6;
X²═NHC(O)R³, NHS(O)₂R³; OC(O)NR⁶R⁷, NHC(O)NR⁶R⁷; and
R⁶, R⁷═H, C₁₋₄ alkyl; 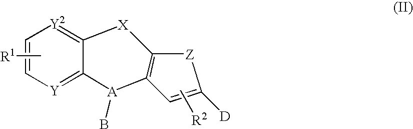
wherein:
Y, Y²═CH, N;
X═—C₂—CH₂—, —CH═CH—, CH₂—S—, 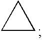
Z=-CH—CH—, —S—;
R¹, R²═Cl, Br, F, CF₃, C₁₋₆ alkyl, C₁₋₆alkylO—;
A=C, CH, N;
provided that when A=CH or N, then B═CH₂—CH₂—CH₂—Y³;
and when A=C, then B═CH—CH₂CH₂—Y³ or 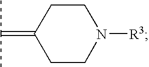
R³═C₁₋₄ alkyl;
Y³═NR⁴R⁵, or 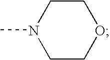
R⁴, R⁵═H, C₁₋₄ alkyl;
Y═X¹—(CH₂)ₙ—X²;
X¹═O, a direct bond;
n=1-6, provided that when X¹ is O, then n=2- 6;
X²═H, OH, OR³, OC(O)R³, NHC(O)R³, NHS(O)₂R³, OC(O)NR⁶R⁷, NHC(O)NR⁶R⁷, C(O)NR⁸R^{9,}
R⁶, R⁷═H, C₁₋₄ alkyl;
R⁸, R⁹═H, C₁₋₄ alkyl, OH, OCH₃, provided that only one of R⁸ and R⁹ can be OH or OCH₃; 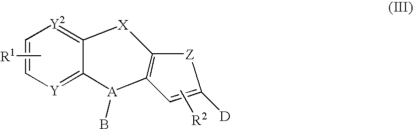
wherein:
Y, Y²═CH, N, provided that at least one of Y and Y² is N;
X═CH₂—O;
Z=-CH—CH—, —S—;
R¹, R²═Cl, Br, F, CF₃, C₁₋₆ alkyl, C₁₋₆ alkylO—;
A=C, CH, N;
provided that when A=N, then B═CH₂—CH₂—CH₂—Y³, and when A=CH then B═ 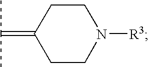
R³═C₁₋₄ alkyl;
Y³═NR⁴R⁵, or 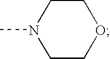
R⁴, R⁵═H, C₁₋₄ alkyl;
Y═X¹—(CH₂)ₙ—X²;
X¹═O, a direct bond;
n=1-6, provided that when X¹ is O, n=2- 6;
X²═H, OH, OR³, OC(O)R³, NHC(O)R³, NHS(O)₂R³, C(O)NR⁶R⁷, OC(O)NR⁶R⁷, NHC(O)NR⁶R⁷, C(O)NR⁸R^{9,}
R⁶, R⁷═H, C₁₋₄ alkyl; and
R⁸, R⁹═H, C₁₋₄ alkyl, OH, OCH₃, provided that only one of R⁸ and R⁹ can be OH or OCH₃.

2. The method of claim 1 wherein the pharmaceutically effective amount of the tricyclic compound is from 0.00001 to 5 wt. %.

3. The method of claim 2 wherein the composition is intended for topical adminsitration to the eye and the pharmaceutically effective amount of the tricyclic compound is from 0.0001 to 0.2 wt. %.

4. The method of claim 3 wherein the pharmaceutically effective amount of the tricyclic compound is from 0.01 to 0.2 wt. %.

5. The method of claim 1 wherein the tricyclic compound is a compound of formula I.

6. The method of claim 1 wherein the tricyclic compound is a compound of formula II.

7. The method of claim 1 wherein the tricyclic compound is a compound of formula III.

8. The method of claim 1 wherein the allergic disease of the eye, ear or nose is selected from the group consisting of allergic conjunctivitis; vernal conjunctivitis; vernal keratoconjunctivitis; giant papillary conjunctivitis; allergic rhinitis; allergic sinusitis; and eustachian tube itching.
